(19) United States
(12) Patent Application Publication
(10) Pub. No.: US 2018/0369552 A1
(43) Pub. Date: Dec. 27, 2018

(54) DEVICE AND METHOD

(71) Applicant: NDM TECHNOLOGIES LIMITED, Loughborough (GB)

(72) Inventors: Dewan Fazlul Hoque CHOWDHURY, Loughborough (GB); Waqas Akbar LUGHMANI, Loughborough (GB); Savvas VOULGARIS, Loughborough (GB)

(73) Assignee: NDM TECHNOLOGIES LIMITED, Loughborough (GB)

(21) Appl. No.: 16/072,481

(22) PCT Filed: Jan. 24, 2017
(86) PCT No.: PCT/GB2017/050175

§ 371 Date: Jul. 24, 2018

(30) Foreign Application Priority Data
Jan. 25, 2016 (GB) 1601288.2

(51) Int. Cl. A61M 37/00

(52) U.S. Cl. CPC A61M 37/0015; A61M 2037/003; A61M 2037/0023

(57) ABSTRACT

A transdermal delivery device comprising a roller, at least one microneedle supported by the roller, and at least one reservoir for a liquid pharmaceutical composition; wherein movement of the roller over the skin of a patient will cause it to rotate and bring the at least one microneedle into contact with the skin of the patient and to bring the at least one microneedle into fluid communication with the at least one reservoir. A method of assembling a transdermal delivery device comprising attaching the at least one microneedle to the device and attaching the at least one reservoir to the device. A method of administering a liquid pharmaceutical composition to a patient using a transdermal delivery device.

The invention relates generally to drug delivery, and specifically relates to devices that deliver pharmacologically active substances transdermally using microneedles.

### BACKGROUND

Syringes and needles are commonly used for the delivery of drugs through the skin. In recent years microneedles have been developed to enable a minimally invasive method of delivering liquid pharmaceutical compositions through the skin. The use of multiple smaller needles allows the liquid to be deposited in multiple locations to minimize the trauma associated with the expansion of intercellular space as liquid is injected into the skin.

Microneedles are a relatively recent invention arising from the application of methods such as etching and lithographic techniques from the semiconductor fabrication processes, to produce sharp high aspect ratio solid or hollow features on materials such as plastics or metals, which are termed “microneedles” because they have dimensions on the micrometre scale.

Microneedles are generally fabricated in arrays, synthesised using etching techniques, such as chemical or physical etching and standard lithographic procedures. The materials used range from silicon to polymers such as PDMS. They generally measure tens of microns to hundreds of microns in length and varying tip diameters, usually less than 10 microns.

Microneedles are suitable for the transdermal delivery of a very wide range of drugs, pharmacologically active agents and therapeutic agents, for both immediate effect and sustained action.

There are a number of advantages associated with the use of microneedles for the delivery of drugs through the skin, i.e. transdermally. The first of these relates to the advantages relating to the mechanism of drug absorption and distribution when administered through the skin, such as avoidance of the first pass metabolism by the liver, and a reduction on side effects, together with the rapid onset of action. Additionally, in this case there are a number of further advantages ranging from the ability to deliver drugs of almost any physicochemical nature, any type of formulation, e.g., liquid, gel, emulsion, or even as a solid whereby the drug could form part of the needle or be used to coat the needle.

Inserting such microneedles in to the skin vertically (i.e. substantially perpendicular to the surface of the skin) poses the issue of rebound from the skin due to the elasticity of the skin. Whilst this is generally not an issue for longer standard needles, it can be an impediment for microneedles where the depth of penetration is smaller, and therefore any rebound from elasticity would reduce the depth of penetration and could, in the worst case where there are multiple needles, result in some of the needles not penetrating the skin at all, leading to the dose being delivered to the top of the skin.

UK patent application GB 2448493 describes a means of administering such needles which leads to the needles entering the skin at an angle. Such needles may have a reservoir interfaced directly to the back of the needle(s), which is compressed after the needles are inserted in to the skin.

The present invention provides an improved transdermal delivery device.

According to the present invention, there is provided a transdermal delivery device comprising a roller, at least one microneedle supported by the roller, and at least one reservoir for a liquid pharmaceutical composition; wherein movement of the roller over the skin of a patient will cause it to rotate and bring the at least one microneedle into contact with the skin of the patient and to bring the at least one microneedle into fluid communication with the at least one reservoir.

Preferably, movement of the roller causes the at least microneedle to move in an arcuate path into contact with the skin of the patient.

Conveniently, the at least one microneedle has a bore which is brought into fluid communication with the at least one reservoir.

Advantageously, the at least one microneedle is connected to a first fluid port, and the at least one reservoir is connected to a second fluid port, and engagement of the first fluid port with the second fluid port brings the at least one microneedle into fluid communication with the at least one reservoir.

Preferably, the first and second fluid ports comprise luer slip or luer lock connections.

Conveniently, the device comprises a guide track to guide the engagement of the first fluid port with the second fluid port.

Advantageously, the guide track guides the movement of the first fluid port towards the second fluid port.

Preferably, the position of the at least one reservoir is fixed before fluid communication with the at least one microneedle.

Conveniently, the at least one microneedle is carried on a belt.

Advantageously, the device further comprises a second roller, and the belt is wrapped around the first and second rollers.

Preferably, the at least one reservoir is configured to release its contents after the at least one microneedle is brought into fluid communication with the at least one reservoir.

Conveniently, the at least one reservoir is configured to release its contents after the at least one microneedle is brought into contact with the skin of the patient.

Advantageously, the at least one reservoir is configured to release its contents upon compression of the at least one reservoir

Preferably, the at least one reservoir comprises a plunger, and the at least one reservoir is configured to release its contents upon movement of the plunger.

Conveniently, the at least one reservoir contains a liquid pharmaceutical composition.

Advantageously, the at least one microneedle and/or the at least one reservoir are detachable.

According to an aspect of the invention, there is provided a kit comprising;
i. a transdermal delivery device as defined above,
ii. at least one detachable microneedle, and
iii. at least one optionally detachable reservoir for a liquid pharmaceutical composition.

According to an aspect of the invention, there is provided a method of assembling a transdermal delivery device as defined above, comprising attaching the at least one microneedle to the device and attaching the at least one reservoir to the device.

Preferably, the at least one reservoir contains a liquid pharmaceutical composition before attachment to the device.

According to an aspect of the invention, there is provided a method of administering a liquid pharmaceutical composition to a patient using a transdermal delivery device as defined above.

Preferably, the method comprises placing the device in contact with the skin of the patient, and moving the roller over the skin of the patient to cause it to rotate and bring the at least one microneedle into contact with the skin of the patient and to bring the at least one microneedle into fluid communication with the at least one reservoir.

The present invention will now be described, by way of example, with reference to the accompanying figures, in which;

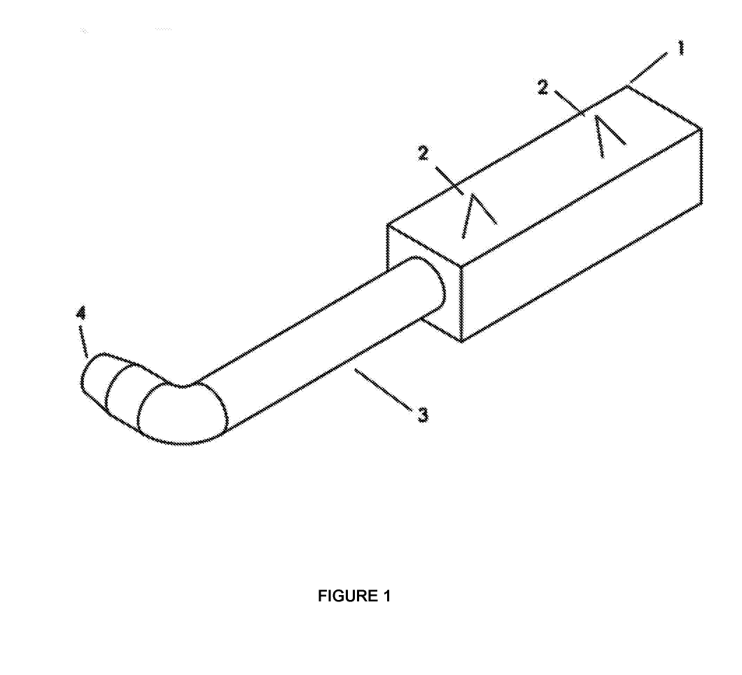

FIG. 1 is a schematic view of a needle container and conduit;

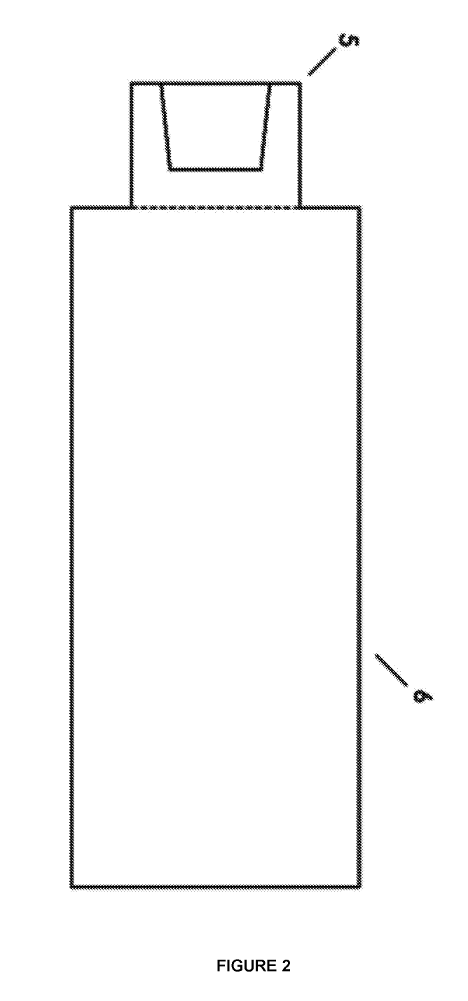

FIG. 2 is a cross sectional view schematic of a drug container;

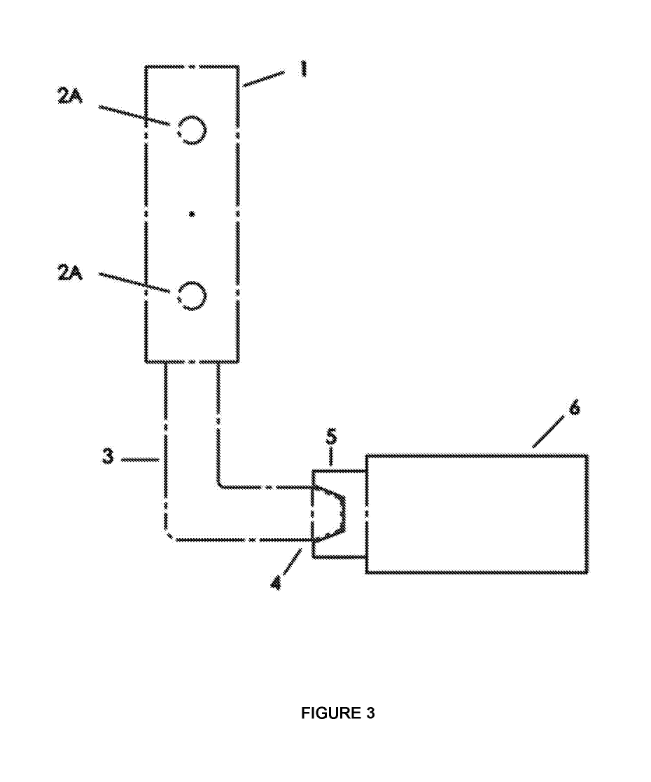

FIG. 3 is a cross sectional schematic view of a needle container interfaced to a drug container;

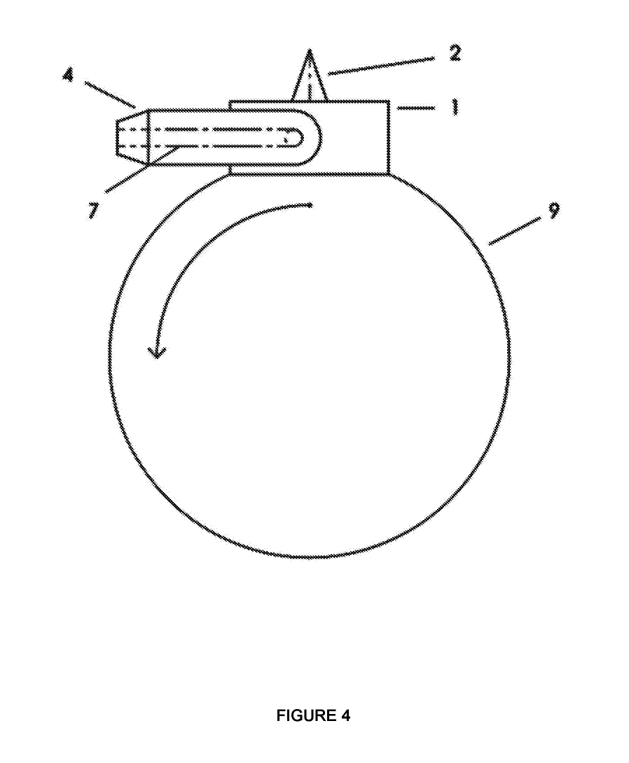

FIG. 4 is a cross sectional schematic view of a needle compartment with a conduit shown positioned on a moveable component, in this case a roller, in a first rest position;

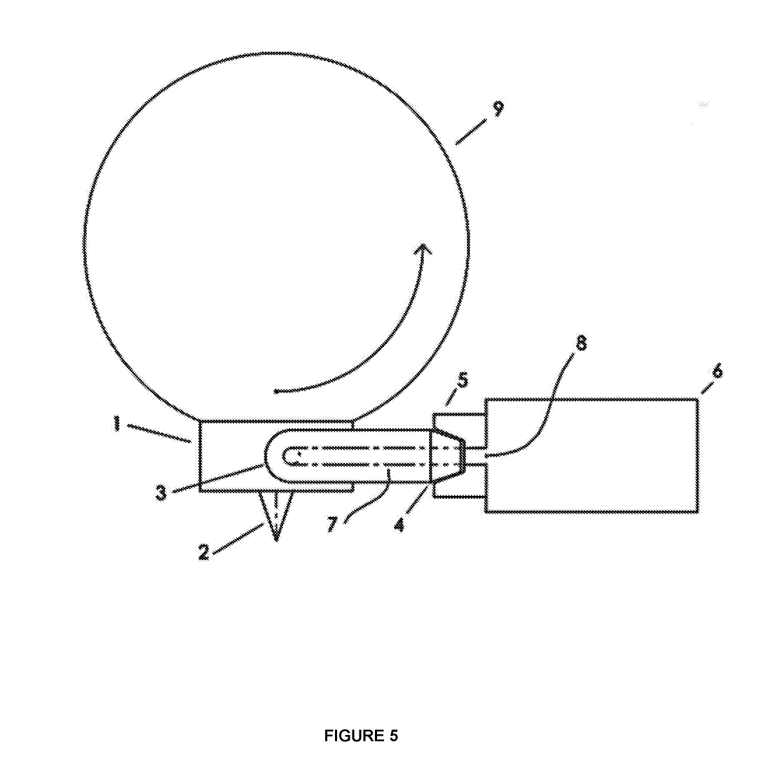

FIG. 5 is a cross sectional schematic view of a needle compartment with a conduit on a moveable roller in a second engaged position, engaged with the drug container;

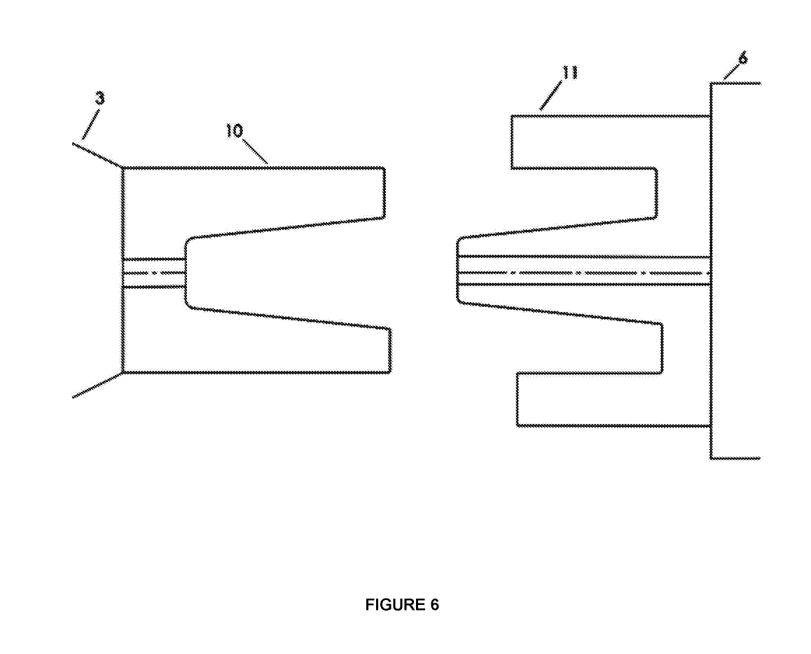

FIG. 6 is an enlarged cross sectional schematic view of the conduit end portion of the drug container and the needle container positioned adjacent to each other;

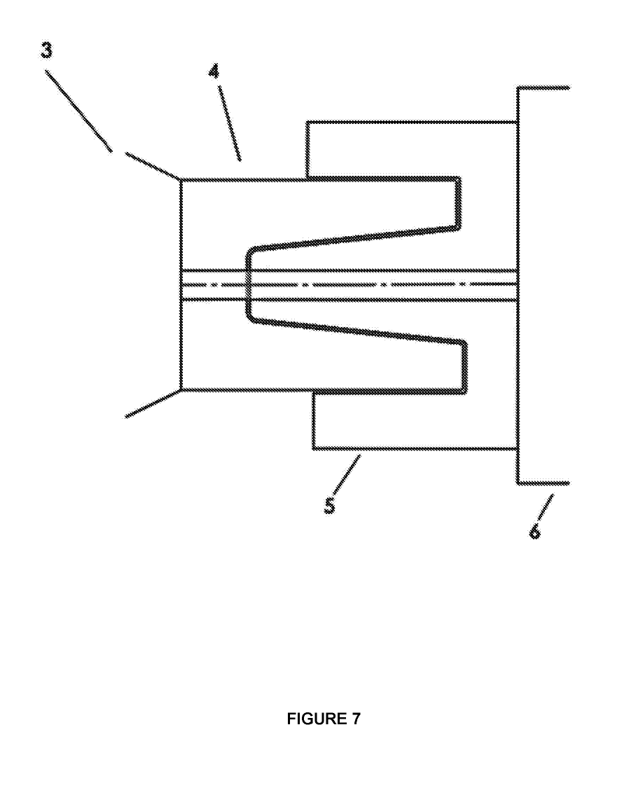

FIG. 7 is an enlarged cross sectional schematic view of the end of the conduits of drug and needle containers in an engaged position;

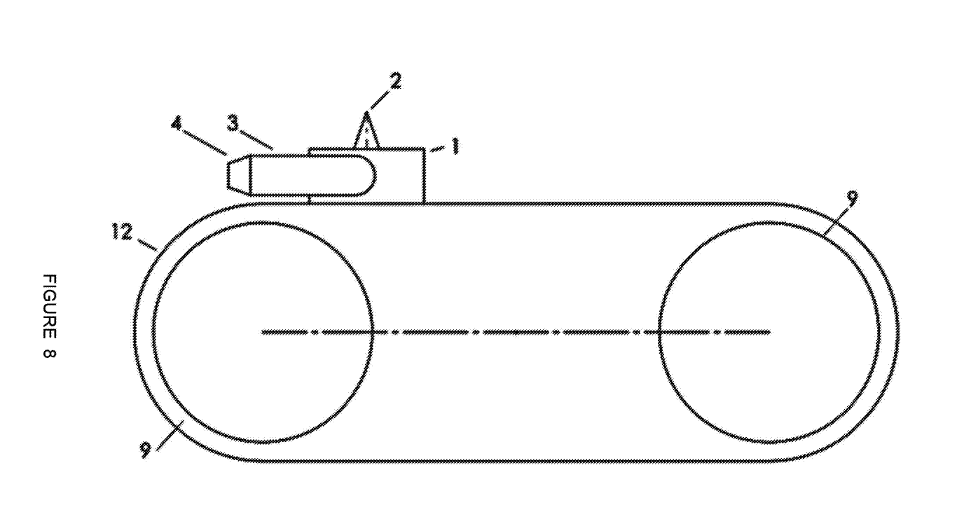

FIG. 8 is a cross sectional schematic view of the needle compartment and conduit shown positioned on a moveable belt mounted on rollers in a first rest position;

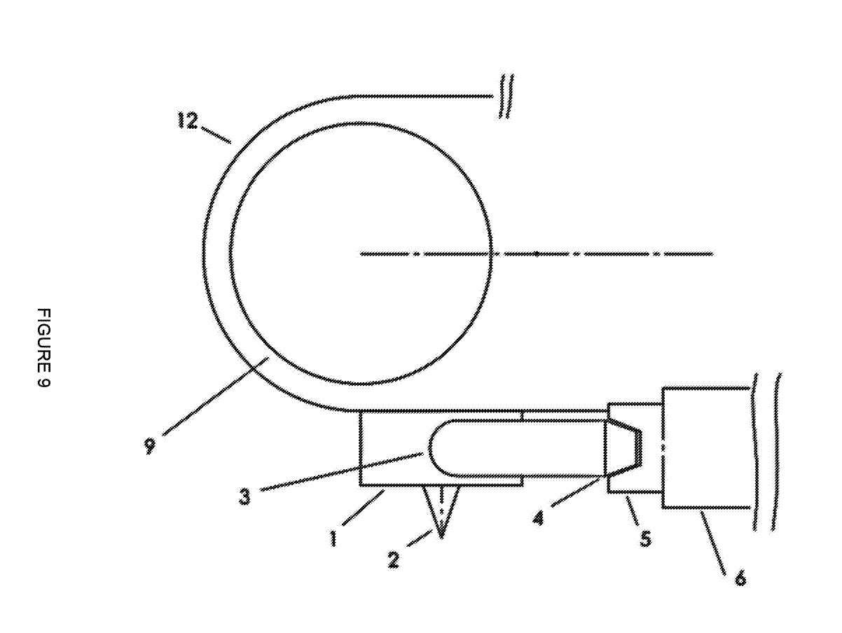

FIG. 9 is an enlarged cross sectional schematic view of the needle compartment and conduit shown positioned on a moveable belt mounted on rollers in a second engaged position whereby the conduit end portion of the needle container is engaged with the end position of the drug container;

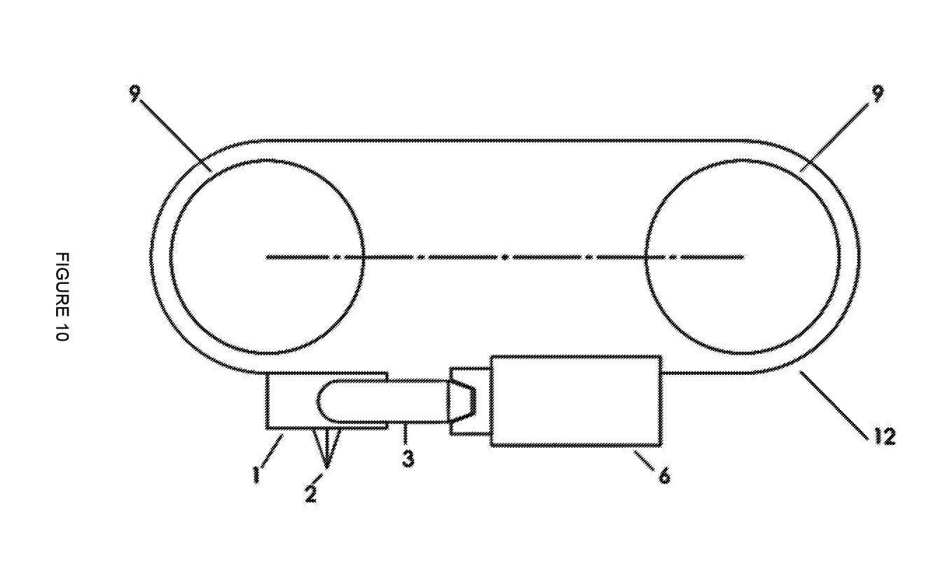

FIG. 10 is an enlarged cross sectional schematic view of the needle compartment and conduit shown positioned on a moveable belt mounted on rollers in a second engaged position whereby the conduit end portion of the needle container is engaged with the end position of the drug container, with both rollers shown;

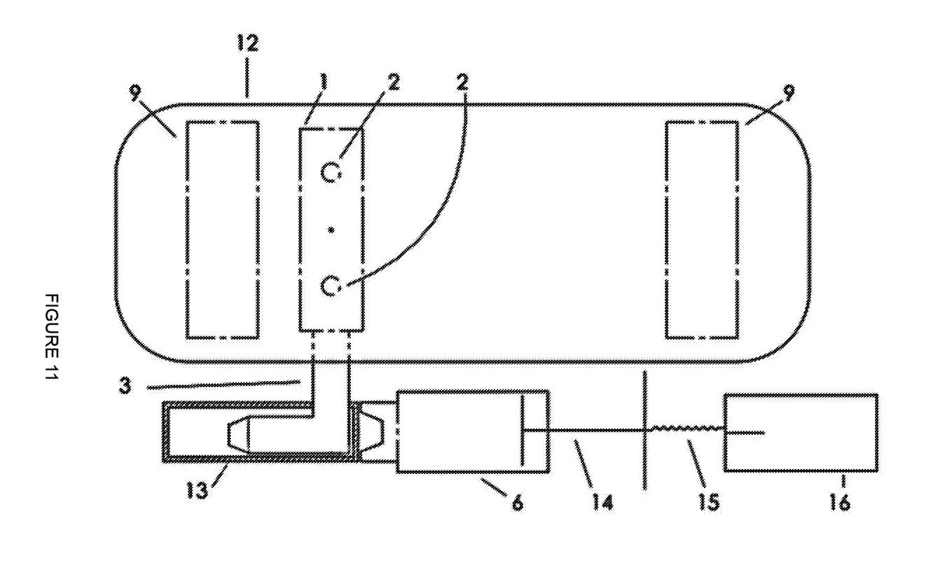

FIG. 11 is a plan view of the needle container in a first rest position mounted on a belt on rollers, and drug container interfaced to an auxiliary energy means;

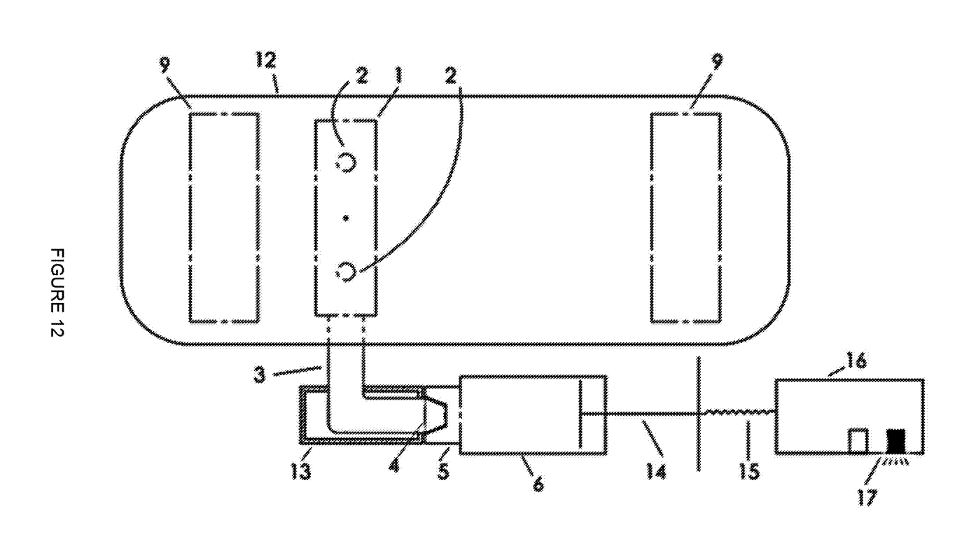

FIG. 12 is a plan view of the needle container in a second engaged position mounted on a belt on rollers, and drug container interfaced to an auxiliary energy means, and indicator means to indicate that engagement between the two containers has occurred;

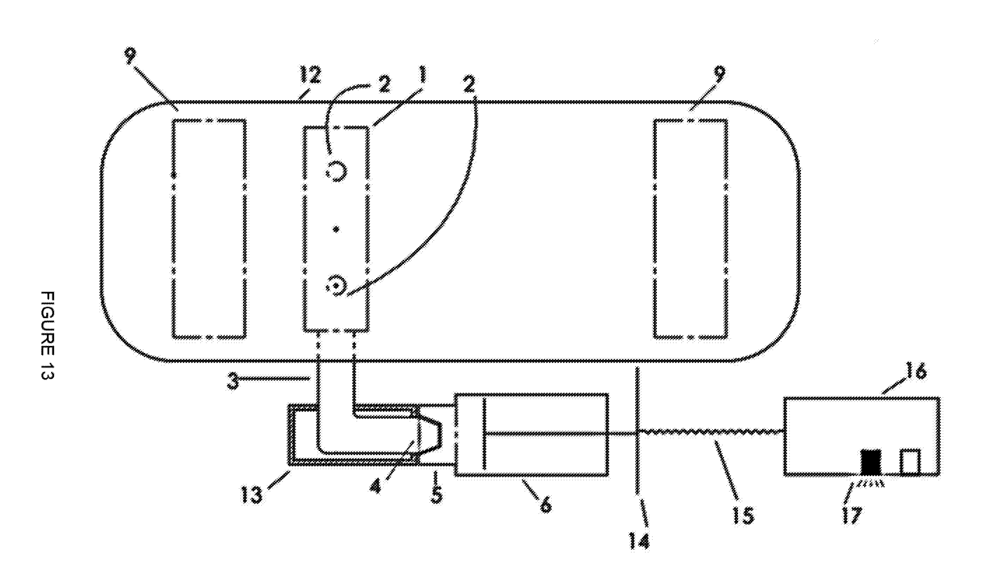

FIG. 13 is a plan view of FIG. 12 showing the indicator in a second mode;

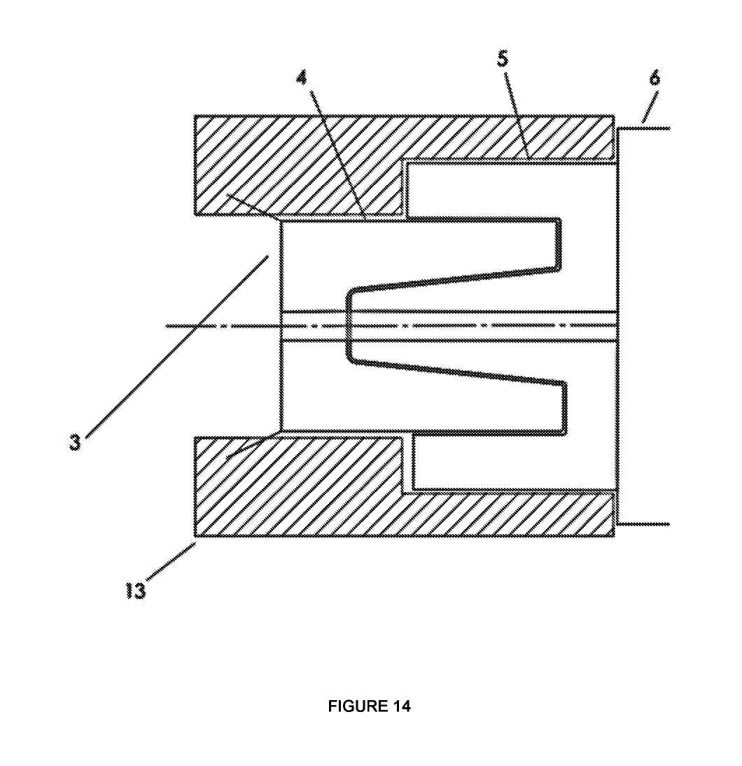

FIG. 14 is a cross sectional view of the engaged needle container end portion and the drug container exit port, within the guide track housing;

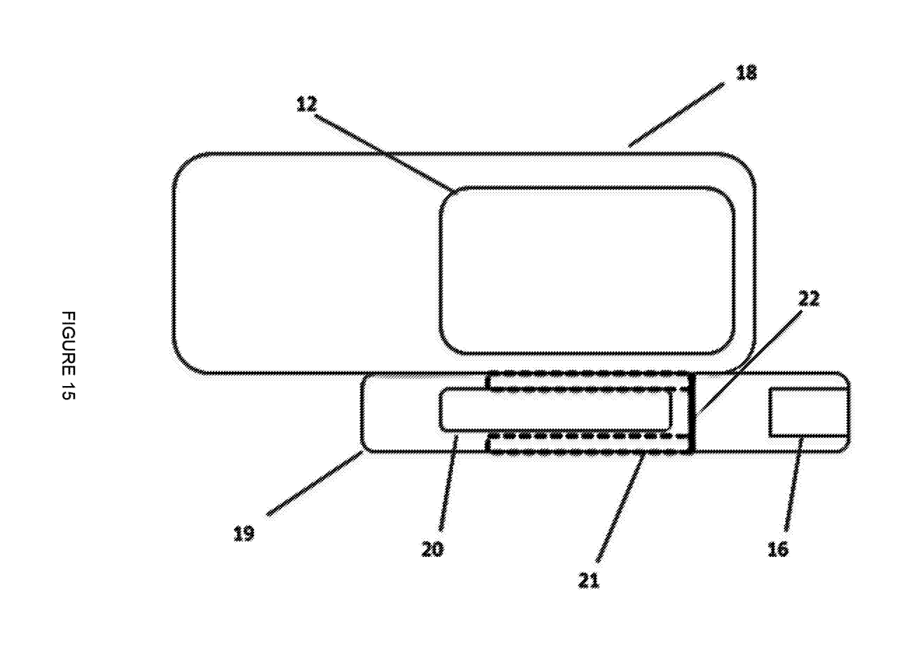

FIG. 15 is a plan view of the outer casing for a roller belt system, housing for the syringe together with back-plate and moveable chamber for the drug container, with the belt system shown in a first rest position; and

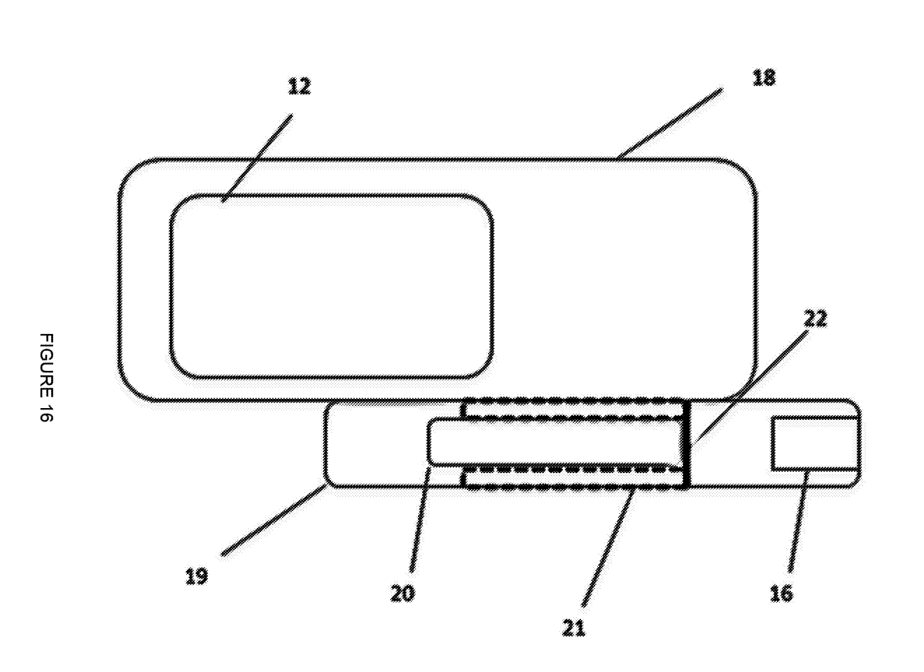

FIG. 16 is a plan view showing the belt system having moved relative to the outer casing to a final activated position, and the drug container housing also moved relative to the moveable chamber within which it is contained.

### DESCRIPTION OF INVENTION

A device and method is described for the administration of drugs through the skin using microneedles. The device is a transdermal delivery device and uses two discrete parts; one part containing the drug and the second part containing the microneedles, in a structural format whereby it allows the flow of liquid from the drug container to the needles and through the bore of the needles into the skin. As will be described, the device enables the microneedles to effectively penetrate the skin of the patient and to deliver a pharmaceutical composition. This is achieved by the coordination of the insertion of the microneedles into the skin with the delivery of a liquid pharmaceutical composition through the microneedles.

The device has one or more suitable microneedles with a bore therethrough. The bore may be a bore running centrally through the microneedle with an exit at or near the tip. The microneedles extend away from a substrate, and may be arranged thereon in regular arrays such as single rows. The orientation and geometry of the microneedles relative to the substrate is preferred such that their penetration through the skin will be enhanced during travel over an arcuate pathway. The rows may extend substantially perpendicularly to the direction of movement of the microneedles. The microneedles may have a generally triangular profile and be arranged so that a sharp leading edge is directed towards the skin for entry into the skin. The bore may exit through the leading edge near the tip.

The microneedles may be made from any suitable materials, ranging from silicon and stainless steel to plastics. Design of the needle will ensure the edge which slices into the skin is sharp and of the optimum geometry to ensure smooth penetration into the skin.

As will be described below, the microneedles may be mounted about a single roller. In a preferred alternative embodiment, each substrate is provided on a closed loop such as a belt or track that runs around one or more rollers to form a conveyor mechanism. It will be understood that the terms “belt”, “track” and “loop” are generally interchangeable.

The roller or rollers may be slidably and rotatably mounted about their axes on a guide on a frame, so that the loop can rotate around the rollers, and the rollers and loop can simultaneously move linearly along the guide as one body.

Preferably the belt is substantially rigid to provide a supporting surface for the microneedles, yet be sufficiently flexible to closely follow the curvature of the rollers.

Rollers in the interstices between the principal rollers can also be provided for additional support. Alternatively, the track may be made in rigid sections joined by a flexible linkage.

The belt may be arranged to move a fixed distance corresponding to a single delivery of drug. The conveyor mechanism may be manually operated, for example by being moved manually across the patient's skin. Alternatively, the conveyor mechanism may be motorised. The mechanism for moving the conveyor along the fixed track may be a simple micro-motor, or a linear actuator such as one produced from shape memory alloy. In either case it is preferred that the pressure distribution is even over the entire body of the patch in contact with the skin.

Where the mechanism is manually operated, e.g. by hand, the device may lack the electronics for driving the microneedles over the skin, or they could be selectively switched off. The device would be housed in a suitable designed casing and driving means would be provided. A manually operated device could be used for single dose administration, e.g., the administration of vaccines. The device can be secured to a limb using a belt/strap to ensure that when the needle is in contact with the skin the pressure is firm and even, and sustained over a period of time to ensure the drug has had time to penetrate the skin completely.

Each roller may be substantially or wholly cylindrical. Each, or the leading roller, may be polyhedral or have a protruding bulbous section. The rollers both support the belt and exert a substantially uniform pressure on the belt.

Each roller may be substantially the same size and shape to remain in contact with the belt such that the lower surface of the belt is parallel to the skin. Alternatively, the rollers may be of differing sizes so that the lower side of the belt has a non-planar topology for example.

The microneedle or microneedles are attached to or form an integral part of a platform or container that has a fluid communication pathway and an attachment means for attachment to a drug container. The microneedles have a bore to allow the flow of fluid. The bores of the microneedles are in fluid communication with a manifold or channels, forming the fluid communication pathway within the platform or container. Preferably, the microneedles are able to travel over an arcuate or curved pathway before engaging with the drug container, either mounted on a roller, or on a belt mounted on rollers.

The reservoir or drug container is constructed such that, on compression of the container, collapse of the container, or compression of the contents of the container, the liquid is forced out of the drug container to the needle container and through the bore of the needles into the skin of the patient. The drug container will not expel its contents until it has fully engaged with the needle container. The drug may exit before, during or after the needles have fully inserted into the skin of the patient, in a predetermined coordinated manner.

It may also be preferable to keep the needle container and the drug container completely separate. This would facilitate aseptic processing of the drug and the reservoir, and the needle container separately and allow a higher degree of versatility in manufacture methods for the system, whereby the drug could potentially be produced in standard pre-filled syringes and then attached to the microneedle system in use.

Movement of the liquid prior to the insertion of the needles into the skin may be preferred in some embodiments to remove air in the system and purge the conduit and needle bore with the liquid pharmaceutical composition.

The needle container will have an attachment means to connect to the reservoir to form a liquid-tight connection, and may comprise, for example, a standard luer slip or luer lock mechanism.

This attachment means may be an extension of the needle container, and may contain a bend or elbow that allows the direct engagement of the needle container with the drug container. As mentioned, the attachment means for forming a fluid communication between the microneedles and the reservoir may comprise a standard luer slip or luer lock mechanism, or may use other standard needle/syringe interlocking mechanism known in the state of the art. It will be appreciated that an elbow may also be provided on the reservoir, leaving the needle container to have a straight connection instead, (this alternative embodiment is not shown in the figures).

Alternatively, there may be a mid section elbow within the device containing the roller(s) to which the needle container and the drug container both interface, thus negating the need for either to have a hollow elbow section. Preferably, this elbow would be disposable due to the need to maintain a sterile fluid flow pathway each time a pharmaceutical composition is administered and to prevent cross contamination.

So, although the device has been described and illustrated with the microneedle platform coming into direct contact with the drug reservoir (via their respective fluid ports), it is also possible for fluid communication to be made indirectly. For example, the microneedles may be moved by the rotation of the roller to come into engagement with a fluid port located on the device, which itself is in fluid communication with the drug reservoir. In this embodiment, the microneedles and platform may be detachable from the device, and the drug reservoir may be detachable from the device. When attached to the device, they can be in fluid communication indirectly with each other via a conduit provided in or on the device. As mentioned above, such a conduit is preferably detachable and disposable when used in a reusable device.

Such a mechanism involves engagement between the microneedle portion and the reservoir portion to bring these parts of the device into fluid communication with each other. One method of ensuring the two end portions of the conduits of the two containers are aligned is by having a housing with a guide track within which the two conduit end portions are positioned. Typically the drug container exit port would be stationary within this housing, and the needle container conduit would be dynamic, and so would move through the housing guide track as the roller or belt is rolled over the skin, and would gradually engage and lock into position whilst minimizing or avoiding any potential warp or misalignment. The guide track would guide the fluid parts of the microneedles and reservoir together.

In an embodiment, the drug container and the needle container would be two discrete units that can be loaded or mounted on to the roller/belt device, in a detachable format. For example, the drug container may be placed into a slot within the casing of the roller mechanism, which may be secured by way of a cover that may be hinged to the casing. The drug container may be loaded on to the belt or roller in a suitable sized slot or compartment and mechanically locked in place or reversibly bonded in place using adhesive on the underside of the needle container.

The term needle container here is used to describe a container, platform or compartment that consists of a minimum of one needle, a cavity (or manifold) that provides a fluid communication path between the bore of the needle and the container (whilst preferably minimizing any dead space by restricting the volume of the cavity) and a conduit that may be a tubular cavity directly interfaced to the main cavity of the needle container, or may be in the form of an opening on the main cavity of the needle container which is shaped such to allow the interlocking with another tubular cavity from a second component, i.e., the drug reservoir, in a seamless leak-proof manner.

The term drug container is used to define any container or reservoir system which can hold a liquid pharmaceutical composition that is capable of being forced through the bore of a needle when the drug container is activated. The activation may be by compression of the container itself or compression of the contents of the container. The energy required to expel the contents of the drug container through to the needle container and into the skin may be provided by the movement of the device across the skin of the patient, and/or may be imparted using an auxiliary energy means. This may be, for example, a compressed gas, a spring mechanism suitably configured, a mechanical force manually applied, or a force applied using a shape memory metal or coil that is electrically activated, with the end result being the partial or full expulsion of the contents of the reservoir to the microneedles.

The device casing may also contain an integral switch which, when depressed, will activate the reservoir ready for use. This could be, e.g., through the release of a compressed spring which then compresses on a piston which forces the contents of the syringe out through the exit port, whilst simultaneously the switch may indicate once the contents are fully expelled by virtue of the movement of the piston which may appear visible once it reaches its end point, or a tab that is attached to the spring and moves in unison with the spring will indicate when the spring has fully extended (though it will be appreciated this mechanism can be applied to other auxiliary energy inputs). The tab may also be then drawn back after removal of the drug container and needle container, to re-set the system, as is done for example with toy pistols.

The dynamic integration of the reservoir with the needle container allows the two units to be manufactured independent of each other, thus reducing the cost burden associated with such system where the two units are fully integrated. The preferred arcuate pathway for the needle container connection means and fluid conduit also ensures minimal patient intervention with respect to the assembly of such a system prior to use. The system as described would require three steps, loading of the needle container, loading of the reservoir, and pressing the activation button whilst holding the device flat to the skin which can be done single handedly. This also allows the system to be pre-loaded, such that at the point of use a single step of placing on the skin and optionally depressing an activation button is all that is required, and this can also be a single process, whereby the activation of the device can occur as a part of the process of placing firmly on the skin and depressing the outer casing to do so.

Furthermore a flat needle application system for liquid drugs (as opposed to the conventional needle and syringe and pen injectors and auto-injectors) would enable the design of a multitude of aesthetic devices that remove the stigma of using medical devices, and make it easier for self-administration. The loading of the device and removal of the disposable reservoir and needle container can also be automated using a suitable loading system.

The term microneedle refers to one or more needle that is designed to deliver drug into the skin from a container to which it is interfaced, whereby it is capable of being interfaced to a flat container, as compared to the vertical devices including the microneedle system developed by Beckton Dickinson whereby whilst the depth of insertion is minimal, the length of the needles is significant, up to 10 mm, to allow the needles to be adequately attached to the syringe, and also to enable substantial pressures to be applied to ensure all the needles penetrate the skin.

The term arcuate pathway is used here loosely to define a dynamic movement of a needle container from a rest position to an engaged position whereby in the latter position it is releasably interlocked with a reservoir for a liquid pharmaceutical composition, and whereby the liquid pharmaceutical composition does not exit the reservoir until the interlocking has taken place, and the drug does not reach the skin until the needle has preferably at least partially entered the skin.

The activation of the drug reservoir may be modulated such that, on engagement of the drug container with the needle container, there is an initial slow exit of liquid to purge the cavity within the drug container of air, and synchronized such that the liquid starts to exit the needle once the needle has entered the skin. The initial purge may be achieved by enabling the interlocking of the needle container and the drug container to occur (in the spatial sense) prior to the needle entering the skin, i.e., as the needle container is still in a dynamic mode and still moving with the belt or roller. This situation may require the drug container to also move with the needle container at that point until the needle has entered the skin. Once the microneedle has penetrated the skin, both the drug container and the needle container will assume rest stationary positions as the drug is expelled from the drug container, via the fluid port of the microneedle plaform, and via the needle bore into the skin.

This movement of the two containers together in unison can be achieved by locating the drug reservoir in a chamber whereby the chamber itself can move relative to the transdermal delivery device for a fixed distance. The overall distance moved by the needle container may be in the range of a few centimeters and the distance that the drug container would need to move in unison with the needle container upon engaging to purge the cavity prior to the needles inserting in the skin need only be a small fraction of this overall distance, for example, in the order of a few percent of the total distance travelled by the needle container, as dictated by the dead volume in the cavity of the needle container.

A liquid pharmaceutical composition is used herein to define any therapeutic, immunological, cosmetic or agent that may be administered to a human or animal subject, in liquid form (including solutions, suspensions, colloids, and emulsions).

FIG. 1 is a schematic view of a container or platform 1 having attached thereto microneedles 2. The microneedles 2 may be constructed from metals, ceramic, plastics, or glass, or biodegradable materials such as carbohydrates. The microneedles 2 each have a bore. The container 1 may also be produced from similar materials, and may be either a separate part or produced as an integral part with the microneedles built-in.

The platform 1 is attached to a conduit 3 with a needle conduit end portion 4. The conduit 3 has a lumen or bore to allow liquid to pass from the end portion 4, through the conduit 3, into the container 1, and through the bores of the microneedles 2. The end portion 4 of the conduit 3 is a fluid port.

The conduit 3 and end portion 4 may also be produced from any one of these materials mentioned above and may be a different material to the needle and/or container, or the entire unit may be fabricated using the same material. The methods of fabrication may include (micro) machining, laser etching, injection molding and micro-replication. In the case that each of the parts are produced separately they may be bonded using adhesives, sonic welding, thermal welding, or other bonding techniques known in the state of the art.

FIG. 2 is a cross sectional schematic view of a reservoir or container 6 for a liquid pharmaceutical composition having an end region or exit port 5. As will be described, the container is designed to expel the liquid pharmaceutical composition out through the fluid port 5 in a coordinated process linked to the movement of the microneedles 2. The drug container may be compressible, it may contain a piston that allows the contents to be forced out like a syringe, or it may be a collapsible structure such as a polymeric sachet or blow fill seal type container when compressed will collapse and allow the contents to flow out via the end conduit/exit port 5.

FIG. 3 is a cross sectional schematic view of a needle container 1 interfaced to a drug container 6, where the needle end portion conduit 4 of the conduit 3 is engaged with the drug container exit port 5. The fluid port 4 of the microneedles 2 and the fluid port 5 of the reservoir 6 are in engagement, and so bring the microneedles into fluid communication with the reservoir. The underside 2A of the needles 2 is shown in the figure. In order for the microneedles and the drug reservoir to be brought into fluid communication as show, the needle container and associated conduit are moved from an initial separated position into this engaged position by the movement of the device across the skin of a patient. This movement of the device causes movement of the microneedles to bring them into contact with the skin, and to penetrate the skin. The movement of the microneedles also brings them into fluid communication with the reservoir, to give the engaged position shown in FIG. 3.

FIG. 4 is a cross sectional schematic view of a needle compartment 1 containing microneedles 2, and the conduit end portion 4, having an inner bore 7, mounted on a roller 9 in an initial rest position. The needle platform 1 may be attached directly to the roller 9, or may be mounted on a belt carried by the roller. The roller may rotate when the device is moved across the skin of a patient, and this rotation carries the microneedles 2 from the initial position towards the skin of the patient. The rotation also brings the microneedles into engagement with the drug reservoir to form fluid communication, when then allows the liquid pharmaceutical composition to flow from the reservoir and through the microneedles into the skin of the patient.

FIG. 5 is a cross sectional schematic view of the needle compartment 1 containing microneedles 2, and conduit 3, with conduit end portion 4, with an inner bore 7, mounted on the roller 9 in a second engaged position. The device reaches this position from the initial position shown in FIG. 4 by the rotation of the roller and associated movement of the microneedles. FIG. 5 shows that the microneedles have been brought into fluid communication with the drug reservoir. The movement of the microneedles has brought the end portion 4 of the conduit 3 into contact and engagement with the exit port 5 of the drug reservoir 6. The fluid port 4 of the microneedles is interfaced with the fluid port 5 of the drug reservoir 6, which comprises a hollow bore 8 creating a fluid communication path between the drug container 6 and needle container 1.

FIG. 6 is an enlarged cross sectional schematic view of one embodiment of the conduit end portion 4 of the conduit 3 of the microneedles and the exit port 5 of the reservoir 6 positioned adjacent to each other. There is a single recessed connection means 10 shown on the end conduit portion 4 of the needle container and a double recessed connection means 11 on the fluid port 5 of the reservoir 6. It will be appreciated these recessed connections are interchangeable between the two fluid ports of the two containers.

As the microneedles are moved by the rotation of the roller, the connection means 10 and 11 are brought into contact with each other, forming a fluid communication. This situation is illustrated in FIG. 7, showing the end portion 4 of conduit 3 of the microneedles 2 in fluid communication with the port 5 of the reservoir 6.

FIG. 8 is a cross sectional schematic view of an embodiment of a transdermal delivery device, with a needle compartment 1, with microneedles 2, and conduit 3, with end portion 4, shown positioned on a moveable belt 12 mounted on first and second rollers 9 in a first rest position. The belt is looped around both of the two rollers 9. The microneedles are shown in an initial position, located away from where the device will initially be placed on the skin of patient. The device also comprises a drug reservoir (not shown in this figure), located in a fixed position relative to the device. As will be explained, the device is placed in contact with the skin of a patient (on the underside of the device as shown in FIG. 8). The device is then firmly pressed against the skin and moved across the skin. This causes rotation of the roller and movement of the microneedles towards the skin. The arcuate movement of the needles is preferred as it brings them into contact with the skin at an angle, allowing for effective penetration, and avoids problems with the resistance to penetration associated with vertical movement of the needles. The movement of the microneedles leads to the situation shown in FIG. 9.

FIG. 9 is a partial enlarged cross sectional schematic view of the needle compartment 1 containing microneedles 2, and conduit 3 with end portion 4, engaged with the exit port 5 of the reservoir 6, shown positioned on a moveable belt mounted on rollers 9 in a second engaged position, in which the conduit end portion of the needle container 4 is engaged with the exit port 5 of the reservoir 6. The movement of the device across the skin of a patient brings the needles 2 into contact with the skin to enable effective penetration (the skin is not shown in this figure). The penetration of the microneedles into the skin is coordinated with the delivery of the liquid pharmaceutical composition. The connection of the microneedles with the source of drug is made at a predetermined position of the microneedles during their movement by the roller. This allows the needles to be properly inserted into the skin before the pharmaceutical composition is administered, and also allows for the reliable delivery of the composition from the reservoir through the needles. The delivery of the liquid pharmaceutical composition from the reservoir can be achieved by a number of different methods, and can be optimized to ensure that the liquid is reliably administered to the patient.

FIG. 10 is an enlarged cross sectional schematic view of the needle compartment 1 containing needle 2, and conduit 3 with end portion 4, engaged with the exit port 5 of the reservoir 6, shown positioned on a moveable belt mounted on rollers 9 in a second engaged position whereby the conduit end portion of the needle container 4 is engaged with the exit port 5 of the drug container 6.

FIG. 11 is a plan view from the underside of the transdermal delivery device. The figure shows the needle container 1 on the top of the device, with microneedles 2, and conduit 3, in a first rest position mounted on a belt 12 on rollers 9, and reservoir 6 in a rest position interfaced to an auxiliary energy means 15, connected to an energy connection and switching compartment 16. The reservoir 6 is mounted at a fixed position near the underside of the device. The needles 2 and reservoir 6 are not in fluid communication in this initial position. The needle container conduit is shown shrouded in a compartment/guide track housing 13, which is a guide track that allows the precise movement of the needle container fluid port during the interfacing and engagement process with the reservoir fluid port. This compartment may be constructed from plastics or metal or ceramic or other material that is known in the state of the art that will provide minimal frictional resistance with fine tolerances during the movement of the needle container conduit over the required pathway, and allow precise engagement with the drug container exit port.

Movement of the device across the skin of the patient leads to the situation shown in FIG. 12, which is a plan view of the needle compartment 1 in a second engaged position with the reservoir 6. The rotation of the rollers 9 has brought the needles 2 around from the initial position of FIG. 11, towards and into the skin of the patient. The needles have also been brought into fluid communication with the drug reservoir. The guide track housing 13 is shown to extend beyond the length of the needle container conduit 3, leading up to the conduit end portion 4, however it will be appreciated that this housing 13 can extend through to also house the fluid port 5 of the reservoir 6. The guide track helps to ensure that the fluid port of the microneedles reliably engages with the fluid port of the drug reservoir.

The auxiliary energy connection and switching compartment 16 is shown to have an indicator 17 in a first mode. The auxiliary energy means 15 is shown interfaced with a plunger 14, which, when activated, will compress the contents of the reservoir 6 and cause the liquid pharmaceutical composition contained therein to be expelled via the exit port 5.

The liquid then flows along the conduit 3 and through the microneedles 2, into the patient. There may be a porous membrane or septum (not shown) between the reservoir 6 and the exterior of the fluid port 5 which prevents the liquid pharmaceutical composition from escaping before use, but which would allow the liquid to pass through when the reservoir is compressed or activated in use.

FIG. 13 is a plan view similar to FIG. 12 showing the auxiliary energy connection and switching compartment indicator in a second activated mode, whereby the plunger 14 is now in the fully activated position, having expelled liquid from the reservoir 6. The reservoir 6 may also move towards the auxiliary energy connection and switching compartment, relative to the outer casing of the roller device (not illustrated here), and the distance of travel may be limited by a mechanical stop/back-plate (not shown). The liquid from the reservoir has been expelled to the microneedles and into the patient.

FIG. 14 is a cross sectional view of the engaged needle container end portion 4 and the drug container exit port 5, within the guide track housing 13, showing that both are accommodated within the housing 13, to ensure precise interlocking and engagement to bring the microneedles 2 and the reservoir 6 into fluid communication.

FIG. 15 is a plan view of another embodiment of the device, having an outer casing 18 for a roller belt system, a drug container housing 19 for a reservoir, together with a back-plate 22 and moveable chamber 20 for the reservoir against the housing inner wall 21, shown relative to the auxiliary energy connection and switching compartment 16, shown positioned within the drug container housing 19. The moveable chamber 20 can move relative to the drug container housing 20 and housing inner wall 21, towards the back-plate 22 where it will come to a permanent stop. This movement will occur by virtue of the motion of the rollers and belt after the needle container has interfaced with the drug container, as it will be pushed further back by the needle container until both the needle container and the drug container come to a permanent stop. This figure shows the belt 12 in a first rest position.

FIG. 16 is a plan view similar to FIG. 15, showing the belt 12 in an activated position, in which the belt (and roller combination) have moved relative to the outer casing 18, this figure also illustrates that the moveable drug container chamber 20 has moved back relative to the housing 19 and inner wall 21, and stopped at the back-plate 22. This arrangement allows for some initial movement or travel of the drug reservoir when it initially engages with the microneedles, which causes controlled relatively slow expulsion of some liquid. The flow of liquid from the reservoir is increased when the movement of the chamber 20 is stopped by the back-plate 22.

1. A transdermal delivery device comprising a roller, at least one microneedle supported by the roller, and at least one reservoir for a liquid pharmaceutical composition; wherein movement of the roller over the skin of a patient will cause it to rotate and bring the at least one microneedle into contact with the skin of the patient and to bring the at least one microneedle into fluid communication with the at least one reservoir.

2. The device according to claim 1 wherein movement of the roller causes the at least microneedle to move in an arcuate path into contact with the skin of the patient.

3. The device according to claim 1 wherein the at least one microneedle has a bore which is brought into fluid communication with the at least one reservoir.

4. The device according to claim 1 wherein the at least one microneedle is connected to a first fluid port, and the at least one reservoir is connected to a second fluid port, and engagement of the first fluid port with the second fluid port brings the at least one microneedle into fluid communication with the at least one reservoir.

5. The device according to claim 4 wherein the first and second fluid ports comprise luer slip or luer lock connections.

6. The device according to claim 4 further comprising a guide track to guide the engagement of the first fluid port with the second fluid port.

7. The device according to claim 6 wherein the guide track guides the movement of the first fluid port towards the second fluid port.

8. The device according to claim 1 wherein the position of the at least one reservoir is fixed before fluid communication with the at least one microneedle.

9. The device according to claim 1 wherein the at least one microneedle is carried on a belt.

10. The device according to claim 9 wherein the device further comprises a second roller, and the belt is wrapped around the first and second rollers.

11. The device according to claim 1 wherein the at least one reservoir is configured to release its contents after the at least one microneedle is brought into fluid communication with the at least one reservoir.

12. The device according to claim 11 wherein the at least one reservoir is configured to release its contents after the at least one microneedle is brought into contact with the skin of the patient.

13. The device according to claim 11 wherein the at least one reservoir is configured to release its contents upon compression of the at least one reservoir.

14. The device according to claim 11 wherein the at least one reservoir comprises a plunger, and the at least one reservoir is configured to release its contents upon movement of the plunger.

15. The device according to claim 1 wherein the at least one reservoir contains a liquid pharmaceutical composition.

16. The device according to claim 1 wherein the at least one microneedle and/or the at least one reservoir are detachable.

17. A kit comprising:
a transdermal delivery device as defined in claim 1,
at least one detachable microneedle, and
at least one optionally detachable reservoir for a liquid pharmaceutical composition.

18. A method of assembling a transdermal delivery device as defined in claim 1, comprising attaching the at least one microneedle to the device and attaching the at least one reservoir to the device.

19. The method according to claim 18 wherein the at least one reservoir contains a liquid pharmaceutical composition before attachment to the device.

20. A method of administering a liquid pharmaceutical composition to a patient using a transdermal delivery device as defined in claim 1 comprising placing the device in contact with the skin of the patient, and moving the roller over the skin of the patient to cause it to rotate and bring the at least one microneedle into contact with the skin of the patient and to bring the at least one microneedle into fluid communication with the at least one reservoir.

21. (canceled)
